(19) United States
(12) Patent Application Publication
(10) Pub. No.: US 2003/0017514 A1
(43) Pub. Date: Jan. 23, 2003

(54) Method for quantitative detection of vital epithelial tumor cells in a body fluid

(76) Inventors: Katharina Pachmann, Bayreuth (DE); Ulrich Pachmann, Bayreuth (DE)

Correspondence Address: Mark S. Ellinger, Ph.D., Fish & Richardson P.C., P.A., Suite 3300, 60 South Sixth Street, Minneapolis, MN, 55402, US

(21) Appl. No.: 10/161,151

(22) Filed: May 31, 2002

(30) Foreign Application Priority Data
Jun. 2, 2001 (DE) DE 101 27 079.8

(51) Int. Cl. C12Q 1/68; G01N 33/574

(52) U.S. Cl. 435/7.23

(57) ABSTRACT

The invention relates to a method for detection of epithelial tumor cells in a body fluid, comprising the following steps:

a) obtaining a defined quantity of a body fluid,

b) labeling the vital epithelial tumor cells by addition, to the body fluid, of antihuman epithelial antibodies which are bound to magnetic particles,

c) labeling the vital epithelial tumor cells by addition, to the body fluid, of antihuman epithelial antibodies which are bound to a fluorochrome,

d) magnetically enriching the vital epithelial tumor cells,

e) immobilizing the suspension so obtained on a support material,

f) recording the vital epithelial tumor cells by means of laser scanning cytometry and calculating the number of these cells in relation to the quantity of body fluid initially obtained.

The invention relates to a method for quantitative detection of vital epithelial tumor cells in a body fluid.

The invention relates generally to the field of the indication of solid tumors. It is well known that metastasis of solid tumors is the main reason for the high mortality rate from cancer. It is caused by cells which are disseminated in the lymph nodes and/or circulate in the peripheral blood. Some of the circulating tumor cells can, under certain circumstances, reach remote compartments where they begin to grow again. In the case of a number of tumors, these compartments are known. In breast cancer and cancer of the colon, one such compartment is the bone marrow. The incidence of the tumor cells in relation to normal bone marrow cells is at most 10⁻³ to 10⁻⁷ tumor cells/normal bone marrow cells. To obtain samples for bone marrow diagnosis, a special procedure is required in combination with or following an operation. Regular monitoring would require repetition of this procedure. Given the inconvenience this causes to the patient and the expenditure in terms of cost and time, it is sought to keep the number of surgical procedures as low as possible.

A further possibility is to examine the peripheral blood, which is much easier to access. However, the problem in this case is that detectable tumor cells in the peripheral blood are present only in extremely small numbers. Another difficulty is that the tumor cells circulating in the peripheral blood can contaminate the transplant in high-dose chemotherapy or autologous peripheral blood stem cell transplantation. Systems with a high level of sensitivity are therefore required to detect such a small number of residual tumor cells.

The most sensitive detection method available at the present time is the polymerase chain reaction (PCR). In the case of hematological malignant growths, the PCR of gene sequences which are associated with the tumor shows a high level of sensitivity in the detection of a small number of tumor cells. However, the use of PCR on solid tumors is associated with problems regarding ease of use, specificity and clinical effect. Tissue-dependent and maturation-dependent expression of surface antigens or intracellular antigens is another method that can be used to immunologically differentiate aberrant cells from normal tissue. However, the incidence of 10⁻³ to 10⁻⁸ with which tumor cells from solid tumors are normally to be expected in peripheral blood necessitates the testing of a large number of negative cells in order to find a positive cell or tumor cell. This type of immunological detection of tumor cells must be carried out with the aid of a microscope. It is very labor-intensive. In this type of detection, tumor cells may be overlooked because of the small number in which they are present. The accuracy of such a method of detection is comparatively low. Moreover, the use of image analysis methods improves the sensitivity and speed only to a small degree.

In known methods, the number of tumor cells is determined in relation to the number of all the cells, for example leukocytes. However, the number of leukocytes can vary greatly, for example in high-dose chemotherapy, so that the conclusions to be drawn from the number of tumor cells in relation to the number of leukocytes are of only limited value.

It is an object of the invention to eliminate the disadvantages of the prior art and in particular to make available an improved method for quantitative detection of vital epithelial tumor cells in a body fluid, the accuracy and speed of which method surpass those of previously known methods.

This object is achieved by the features of claim 1. Advantageous embodiments of the invention are evident from the features set out in claims 2 to 11.

The invention provides a method for quantitative detection of vital epithelial tumor cells in a body fluid, comprising the following steps:

a) obtaining a defined quantity of a body fluid,

b) labeling the vital epithelial tumor cells by addition, to the body fluid, of antihuman epithelial antibodies which are bound to magnetic particles,

c) labeling the vital epithelial tumor cells by addition, to the body fluid, of antihuman epithelial antibodies which are bound to a fluorochrome,

d) magnetically enriching the vital epithelial tumor cells,

e) immobilizing the suspension so obtained on a support material,

f) recording the vital epithelial tumor cells by means of laser scanning cytometry and calculating the number of these cells in relation to the quantity of body fluid initially obtained.

The proposed method affords the possibility of determining the number of vital epithelial tumor cells directly in a body fluid, for example blood, bone marrow, bone marrow aspirate, transudate, exudate, lymph, apheresis fluid, ascites, urine, saliva, and drainage fluids from wound secretions. The antibodies used bind specifically to vital cells suspected to be tumorous. Vital tumor cells can thus be separated from dead tumor cells. The number of vital tumor cells can be given in relation to the volume of the body fluid used. The method according to the invention supplies standardized values. In addition, the support material on which the body fluid is applied after labeling, enrichment and separation can be stored for documentation purposes, so that it is available for later evaluation and further characterization. By contrast, in the previous methods, only one measurement protocol can be stored.

With the method according to the present invention, it is possible to detect very small quantities of tumor cells in a body fluid. For test purposes, for example, ten tumor cells were added to 20 ml of whole blood. It was possible to detect all of these ten tumor cells by the method according to the invention. This result is comparable to that which can be obtained by means of PCR, but the disadvantages associated with the latter method can be avoided. The method according to the invention permits quantitative determination of vital epithelial tumor cells in a body fluid.

Prior to the labeling of the vital epithelial tumor cells with antibodies, the body fluid, in particular peripheral blood, is advantageously lyzed in order to separate off erythrocytes, for example. The suspension obtained is then centrifuged and the supernatant is separated off and discarded. It is not necessary for all the erythrocytes to be removed since they do not influence the method.

In a variant of the invention, prior to magnetic enrichment, the tumor cells are labeled both with antihuman epithelial antibodies which are bound to magnetic particles (magnetic beads or microbeads), and with antihuman epithelial antibodies which are bound to a fluorochrome. The magnetic particles preferably have a diameter smaller than 70 nm. The tumor cells carry both magnetic particles and a fluorescent dye via the antigen-antibody binding. The tumor cells are then enriched by magnetic cell separation by being placed on a column, for example, which is located in a strong magnetic field, e.g. formed by a permanent magnet. The cells of the body fluid to which no magnetic particles are bound are flushed out of the column. The labeled cells remain in the column as a result of the action of the magnetic field. After the magnetic field is removed, the tumor cells remaining in the column can be flushed out.

Alternatively, the magnetic cell separation can take place before the vital epithelial tumor cells are labeled by addition of antihuman epithelial antibodies which are bound to a fluorochrome. For this, it is sufficient if the tumor cells are labeled with the magnetic particles.

An FcR blocking reagent is advantageously added to the body fluid prior to the labeling of the vital epithelial tumor cells.

The antibodies used are preferably antihuman epithelial antibodies (HEA) from mice. The fluorochrome used is preferably fluorescein isothiocyanate (FITC).

After enrichment and staining of the vital epithelial tumor cells, the cell suspension is placed on a support. This is advantageously a glass slide which is preferably coated with poly-L-lysin.

The number of tumor cells on the support or support material is determined by means of laser scanning cytometry. For outlining the cells located on the support material, the forward scatter is advantageously used as threshold parameter at low magnification. The background fluorescence can be determined dynamically in order to determine the maximum fluorescence intensity and/or the total fluorescence by integration over each cell. This makes it possible to correct changes in the background fluorescence, so that the fluorescence can be calculated under the same conditions for each cell and equivalent results can be obtained for each cell. The green fluorescence of the FITC-HEA-labeled cells is preferably recorded using a 530/30 nm bandpass filter.

The enriched and FITC-labeled cells (FITC-positive cells) can additionally be stained with a further fluorescent dye. For example, the DNA of the cell nuclei can be stained with a DNA-specific dye such as propidium iodide. The red fluorescence of the cells stained in this way is likewise recorded by means of laser scanning cytometry using a 625/28 nm bandpass filter. The forward scatter is used as the threshold parameter for outlining. The measured red and green fluorescence are compared with one another and the number of positive results is determined.

The number of labeled positive cells per volume is particularly high, compared to the method known in the prior art, because of the enrichment, so that the speed and accuracy of the method according to the invention are considerably greater. The number of labeled cells found is then referred back to the volume of body fluid initially used. This means it is possible to compare detection results determined at different times. The success of tumor therapy, for example, can be quickly ascertained.

The morphology of the positive cells recorded can then be determined by hematological staining methods, for example the May-Grünwald stain.

The method according to the invention advantageously allows a number of quantitative detections to be carried out one after another. The cells can be examined quantitatively with respect to various parameters. For this purpose, after a first quantitative assessment, the cells can be treated, for example, with a solution containing a second detection substance, e.g. a fluorescent dye, which is specific for malignancy. The coordinates of the cells are already known and stored in a computer before the first quantitative detection is carried out. Each cell can be immediately located and its reaction to other detection substances recorded and quantitatively evaluated. Further detection methods can be carried out, in particular FISH or TUNEL, and quantitatively assessed.

The invention is explained in more detail below on the basis of illustrative embodiments.

### EXAMPLE 1

In this example, a method for detection of epithelial tumor cells in blood is described in which the magnetic enrichment of the vital epithelial tumor cells takes place after the labeling of the vital epithelial tumor cells by addition of antihuman epithelial antibodies to which a flurorochrome is bound.

a. Preparation of a Dilution Series of Whole Blood Containing Tumor Cells

Peripheral blood was mixed with different proportions of tumor cells. The tumor cells were derived from a breast cancer cell line (type SK Br2). The leukocytes of the peripheral blood and the tumor cells were counted. In a first dilution step, 6×10⁵ tumor cells were mixed with 100 μl of whole blood which contained 6×10⁵ leukocytes. In the next dilution step, 6×10⁴ tumor cells and 6×10³ tumor cells were each added to 100 μl of blood. In the next dilution step, 6×10³ and 6×10² tumor cells were added to 1 ml of blood, and 6×10² and 60 tumor cells were added to 10 ml or 20 ml of blood, in the last case a dilution of 5 tumor cells to 107 normal cells was obtained.

b. Magnetic Enrichment and Staining

For separation on a magnetic column, 400 μl with up to 5×10⁷ cells from the dilution series obtained in section a. were incubated with 100 μl of blocking reagent in the cold for 30 minutes. The cells were then treated with magnetic particles to which antihuman epithelial antibodies are bound (100 μl HEA microbeads, Milteny), incubated for 15 minutes and washed 10 times with the labeling buffer. The columns on which a magnet is arranged were washed and the magnetic particles were collected in the columns. The negative cells were then eluted by flushing with 5×500 μl of buffer and the columns were separated from the magnet. The cells remaining in the columns were flushed out with additional buffer and stained with 50 μl of FITC-conjugated antihuman epithelial mouse antibodies (HEA-FITC-antibodies, Milteny).

c. Immobilizing the Cells and Determining the Number of Cells

The cells were placed on slides. After applying 100 μl of the cell suspension, the vital cells adhered to the surface of the slide after 10 to 15 minutes. For optimal measurements, an individual cell suspension must be applied to the slide with the cells being spaced apart from one another by a distance which is approximately 2 to 3 times the diameter of a cell.

The adhering cells were measured using a laser scanning cytometer (LSC Compucyte Corp.). The outlining of the cells adhering to the slide was carried out using the forward scatter as threshold parameter at 20× magnification. The background fluorescence was determined dynamically in order to calculate both the maximum fluorescence intensity and the total fluorescence on the basis of one cell. This makes it possible to correct variations in the background fluorescence so that the calculation of the fluorescence takes place the same way for all the cells. The green fluorescence of the positive HEA-FITC cells was recorded with a 530/30 nm bandpass filter and strengthened with a photomultiplier.

The cells on the slides were then centrifuged and immersed in a PBS solution containing 1 mg/ml propidium iodide. The threshold value for the outlining of the cells was likewise based on the forward scatter. The red fluorescence was recorded with a 625/30 nm bandpass filter and strengthened with a second photomultiplier. The red and the green fluorescence were compared using a computer program (WinCyte, Compucyte Corporation) and represented as scatter diagrams, histograms, percentages and means of the FITC-positive and FITC-negative cells, the calculation being based only on the area which includes individual cells.

d. Result

The correlation between the calculated number of tumor cells in a sample of the dilution series and the number determined by the method was very high (>0.99). Even at the 10⁻⁸ dilution, it was possible to detect 50 of 60 cells.

### EXAMPLE 2

In this example, a method for detection of epithelial tumor cells in blood is described in which the magnetic enrichment of the vital epithelial tumor cells takes place prior to the labeling of the vital epithelial tumor cells by addition of antihuman epithelial antibodies to which a flurorochrome is bound.

a. Labeling of the Tumor Cells

20 ml of peripheral blood was combined with 40 ml of erylysis buffer (155 mM NH₄Cl, 10 mM KHCO₃, 1 mM Na₂-EDTA) and centrifuged cold at 2000 rpm for 7 minutes. The supernatant was discarded and the sediment was taken up in PBS-EDTA solution (50 ml of PBS with 200 μl of 0.5 M EDTA), so that the total volume was 900 μl. 100 μl of FcR blocking reagent (Milteny), 100 μl of the magnetic particles to which antihuman epithelial antibodies are bound (100 μl of HEA microbeads, Milteny) were added to 300 μl of this mixture and mixed with it. 50 μl of FITC-conjugated antihuman epithelial mouse antibodies (HEA-FITC antibodies, Milteny) were then added to this suspension and incubated cold for 15 minutes.

b. Magnetic Enrichment

The separation columns were placed in the magnets (ctaMACS, Milteny) and washed twice with 500 μl of PBS-EDTA solution. The suspension obtained in section a. was taken up with 500 μl of PBS-EDTA solution and applied to the column. The column was washed three times with 500 μl of PBS-EDTA solution and removed from the magnet. The column was then flushed with 200 μl of PBS-EDTA solution in order to take up the labeled cells.

c. Immobilizing the Cells and Determining the Number of Cells

100 μl of the solution obtained in section b. were applied to and spread evenly across a slide (Schubert Laboratories) coated with poly-L-lysin. The number of tumor cells was determined as in example 1, section c, and the number was referred to the initially used volume of peripheral blood.

1. Method for quantitative detection of vital epithelial tumor cells in a body fluid, comprising the following steps:
a) obtaining a defined quantity of a body fluid,
b) labeling the vital epithelial tumor cells by addition, to the body fluid, of antihuman epithelial antibodies which are bound to magnetic particles,
c) labeling the vital epithelial tumor cells by addition, to the body fluid, of antihuman epithelial antibodies which are bound to a fluorochrome,
d) magnetically enriching the vital epithelial tumor cells,
e) immobilizing the suspension so obtained on a support material,
f) recording the vital epithelial tumor cells by means of laser scanning cytometry and calculating the number of these cells in relation to the quantity of body fluid initially obtained.

2. Method according to claim 1, in which laser scanning cytometry is carried out to determine the maximum fluorescence intensity and/or the total fluorescence per cell.

3. Method according to one of the preceding claims, in which the background fluorescence is dynamically determined so that equivalent fluorescence values are obtained for each cell.

4. Method according to one of the preceding claims, in which the magnetic enrichment of the vital epithelial tumor cells takes place prior to the labeling of the vital epithelial tumor cells by addition, to the body fluid, of antihuman epithelial antibodies which are bound to a fluorochrome.

5. Method according to one of the preceding claims, in which the magnetic enrichment of the vital epithelial tumor cells takes place after the labeling of the vital epithelial tumor cells by addition, to the body fluid, of antihuman epithelial antibodies which are bound to a fluorochrome.

6. Method according to one of the preceding claims, in which, prior to the labeling of the vital epithelial tumor cells with antibodies, the body fluid is lyzed and centrifuged and the supernatant separated off and discarded.

7. Method according to one of the preceding claims, in which a blocking reagent is added to the body fluid prior to the labeling of the vital epithelial tumor cells.

8. Method according to one of the preceding claims, in which antihuman epithelial antibodies from mice are used to label the vital epithelial tumor cells with antihuman epithelial antibodies.

9. Method according to one of the preceding claims, in which fluorescein isothiocyanate (FITC) is used as fluorochrome.

10. Method according to one of the preceding claims, in which the body fluid is chosen from the following group: blood, bone marrow, bone marrow aspirate, transudate, exudate, lymph, apheresis fluid, ascites, urine, saliva, and drainage fluids from wound secretions.
